(19) United States
(12) Patent Application Publication
(10) Pub. No.: US 2013/0216530 A1
(43) Pub. Date: Aug. 22, 2013

(54) Methods for optimizing biological response modifier therapy using therapeutic drug monitoring of immunosuppressants

(71) Applicant: Exagen Diagnostics, Inc., (US)

(72) Inventor: Thierry Dervieux, San Diego, CA (US)

(73) Assignee: Exagen Diagnostics, Inc., Albuquerque, NM (US)

(21) Appl. No.: 13/756,616

(22) Filed: Feb. 1, 2013

Related U.S. Application Data
(60) Provisional application No. 61/593,918, filed on Feb. 2, 2012.

(51) Int. Cl. A61K 39/395; A61K 31/519

(52) U.S. Cl. CPC A61K 39/3955; A61K 31/519
USPC 424/133.1; 424/158.1; 435/7.8

(57) ABSTRACT

The invention provides methods for treating humans in need of combined immunosuppressant and biological response modifier therapy.

### CROSS REFERENCE

This application claims priority to U.S. Provisional Patent Application Ser. No. 61/593,918 filed Feb. 2, 2012, incorporated by reference herein in its entirety.

### BACKGROUND

Methotrexate and azathiopurine form the cornerstone treatment of rheumatoid arthritis and (RA) inflammatory bowel disease respectively (IBD)). Yet approximately 40% of patients affected by these autoimmune diseases do not respond these first-line anti metabolites based therapy. The addition of biological response modifiers (BRMs) together with methotrexate and azathiopurine is becoming the standard of care for RA and IBD treatment. Despite the justified enthusiasm with these monoclonal antibodies (including TNF blockers, infliximab, etanercept, and adalimumab) owing to their rapid onset of action and improvements in quality of life, BRM therapy is associated with immunogenic reactions and formation of autoantibodies that result in loss of efficacy and increase occurrence of side effects.

Currently, there is no method to optimize methotrexate (or other immunosuppressant, including but not limited to leflunomide and azathiopurine) dose in the context of BRM therapy, to help reduce BRM immunogenicity and improve patient outcome.

### SUMMARY OF THE INVENTION

In a first aspect, the present invention provides methods for treating a human subject in need of combined immunosuppressant and biological response modifier (BRM) therapy, comprising
(a) determining a metabolite level of an immunosuppressant in a sample from a human subject receiving or to receive a BRM therapy in combination with the immunosuppressant;
(b) comparing the metabolite level in the sample to a threshold metabolite level below which the BRM leads to unacceptable immunogenicity; and
(c) administering to the human subject a subsequent dose of immunosuppressant and/or a dose of the BRM in an amount effective to treat the subject based upon comparing the metabolite level in the sample to the threshold metabolite level.

In a second aspect, the present invention provides methods for optimizing dosage of an immunosuppressant comprising

(a) determining a metabolite level of an immunosuppressant in a sample from a human subject receiving or to receive a BRM therapy in combination with the immunosuppressant;

(b) comparing the metabolite level in the sample to a threshold metabolite level below which the BRM therapy leads to unacceptable immunogenicity; and

(c) recommending adjustment or adjusting a subsequent dose of immunosuppressant and/or biological response modifier to be administered to the human subject based upon comparing the metabolite level in the sample to the threshold metabolite level.

### DESCRIPTION OF THE FIGURES

FIG. 1 Effects of RBC MTXPG₃ on Infliximab pharmacokinetics. Panel A: Dose normalized infliximab levels (in μg/ml per mg infliximab every 8 weeks) as a function of RBC MTXPG₃. The incidence of ATI for each category of MTXPG₃ level is indicated. 4 patients presented with MTXPG₃<5 nmol/L and 3 of them had ATI. Median MTX dose in patients with RBC MTXPG₃ below 25 nmol/L was 10 mg/week (interquartile range: 7.5.-15 mg/week), it was 15 mg/week (interquartile range: 12.5-17.5 mg/week) in those with RBC MTXPG₃ ranging from 25 to nmol/L and 20 mg/week (interquartile range: 13.7-23.7 mg/week) in those with RBC MTXPG₃>50 nmol/L. Panel B: RBC MTXPG₃ in patients with (n=50) or without (n=11) ATI. Bars represent median with interquartile range and min max values.

### DETAILED DESCRIPTION OF THE INVENTION

All references cited are herein incorporated by reference in their entirety. As used herein, the singular forms “a”, “an” and “the” include plural referents unless the context clearly dictates otherwise. “And” as used herein is interchangeably used with “or” unless expressly stated otherwise. All embodiments within and between different aspects of the invention can be combined unless the context clearly dictates otherwise. As used herein, “about” means +/−5% of the recited range or value.

In a first aspect, the present invention provides methods for treating a human subject in need of combined immunosuppressant and biological response modifier (BRM) therapy, comprising
(a) determining a metabolite level of an immunosuppressant in a sample from a human subject receiving or to receive a BRM therapy in combination with the immunosuppressant;
(b) comparing the metabolite level in the sample to a threshold metabolite level below which the BRM leads to unacceptable immunogenicity; and
(c) administering to the subject a subsequent dose of immunosuppressant and/or a dose of the BRM in an amount effective to treat the subject based upon comparing the metabolite level in the sample to the threshold metabolite level.

As demonstrated in the examples that follow, the inventor has discovered that the immunosuppressant metabolite levels impact BRM exposure in the patient, and thus that treating the subject based on immunosuppressant levels can be used to limit immunogenicity caused by the BRM therapy, such as limiting the development/titer of antibodies against the BRM therapeutic being used to treat the subject. Thus, the methods provide a significant improvement in treatment for patients in need of combined immunosuppressant/BRM co-therapy and can, for example, help to reduce the dosage of BRM administered to the subject.

The methods of this aspect of the invention can be used with any suitable human subject in need of combined immunosuppressant and BRM therapy to treat a disorder, such as those human subjects suffering from an autoimmune disease. The term “autoimmune disease” refers to a disease or disorder resulting from an immune response against a self-tissue or tissue component and includes a self-antibody response or cell-mediated response. The term autoimmune disease, as used herein, encompasses organ-specific autoimmune diseases, in which an autoimmune response is directed against a single tissue, such as Type I diabetes mellitus, myasthenia gravis, vitiligo, Graves' disease, Hashimoto's disease, Addison's disease, autoimmune gastritis, and autoimmune hepatitis. The term autoimmune disease also encompasses non-organ specific autoimmune diseases, in which an autoimmune response is directed against a component present in several or many organs throughout the body. Such autoimmune diseases include, for example, systemic lupus erythematosus, progressive systemic sclerosis and variants, polymyositis, and dermatomyositis. Additional autoimmune diseases include, but are not limited to, pernicious anemia, primary biliary cirrhosis, autoimmune thrombocytopenia, Sjogren's syndrome, rheumatoid arthritis, and multiple sclerosis. In one embodiment, the subject has rheumatoid arthritis. The subject may have had the disease for any length of time prior to treatment with the methods of the invention; in exemplary embodiments, the subject has had the disease for at least 6 months, 1 year, 2 years, 3 years, 5 years, 6 years, or more

In a further embodiment, the subject is an adult, for example, an adult at least 40 years old, at least 50 years old, at least 54 years old, or at least 60 years old. In another embodiment, the subject is female.

As used herein. “treating” means accomplishing one or more of the following: (a) reducing or eliminating the disorder, such as an autoimmune disease, in the subject; (b) reducing the severity of one or more symptoms of the disorder; (c) limiting or preventing development of one or more symptoms of the disorder; (d) inhibiting worsening of one or more symptoms of the disorder; and (e) limiting or preventing recurrence of one or more symptoms of the disorder in subjects that were previously symptomatic for the relevant symptom.

The term “sample” refers to any biological specimen obtained from a subject. Samples include, without limitation, whole blood, plasma, serum, buccal cells, red blood cells, white blood cells (e.g., peripheral blood mononuclear cells), saliva, urine, stool (i.e., feces), tears, any other bodily fluid, a tissue sample (e.g., tumor tissue) such as a biopsy of a tumor, and cellular extracts thereof. In certain instances, the sample is whole blood, serum, or plasma. In one embodiment, the sample is a red blood cell sample.

The methods may comprise the use of any suitable immunosuppressant, including but not limited to methotrexate, leflunomide, azathiopurine, JAK kinase inhibitors, and spleen kinase inhibitors. The methods comprise treating subjects that are already undergoing immunosuppressant therapy. The subject may have been undergoing immunosuppressant therapy for any length of time, so long as levels of the immunosuppressant metabolite can be measured in the sample. In various non-limiting embodiments, the methods are carried out on subjects that have been undergoing immunosuppressant therapy for at least 1 month, 2, months, 3 months, 4 months, 6 months, 1 year, 2 years, 3 years, 4 years, 5 years, or more. Similarly, the methods of the invention can be carried out on patients undergoing any type of immunosuppressant dosing regimen. In various non-limiting embodiments, the methods are carried out on subjects that are undergoing immunosuppressant therapy on a daily basis, every other day, twice a week, weekly, or any other dosing schedule at any suitable dose as determined by an attending physician.

The metabolites can be detected using standard methods in the art. For example, when the immunosuppressant is methotrexate (MTX), the metabolite may be methotrexate polyglutamate (MTXPG), the MTXPG level can be determined, for example, by an assay selected from the group consisting of radiochemical assays, liquid chromatography, enzymatic dihydrofolate reductase inhibitor assays and fluorescent polarization immunoassays.

In embodiments wherein the immunosuppressant is MTX, the metabolite may be MTXPG. The term “methotrexate polyglutamate” is synonymous with “MTXPG” and refers to a derivative of methotrexate having two or more glutamates which are amide bonded to the p-aminobenzoyl moiety of methotrexate. The number of glutamates in a methotrexate polyglutamate varies from two to seven or more; the number of glutamate moieties can be denoted by “n” using the nomenclature MTXPGₙ such that, for example, MTXPG₂ is MTXPG having two glutamates, MTXPG₃ is MTXPG having three glutamates, MTXPG₄ is MTXPG having four glutamates, MTXPG₅ is MTXPG having five glutamates, MTXPG₆ is MTXPG having six glutamates, MTXPG₇ is MTXPG having seven glutamates, and MTXPG₃₋₅ is a mixture containing MTXPG₃, MTXPG₄, and MTXPG₅, with the ratio of the individual polyglutamated forms in the mixture not defined. In certain embodiments, a level of one or more long-chain MTXPG species is determined by resolving them from short-chain MTXPGs and other molecules. As used herein, the term “long-chain MTXPG” refers to any MTX having at least three glutamates attached thereto. In these embodiments, it is further preferred that the sample comprise red blood cells (ie: red blood cells or extracts derived from red blood cells). In one embodiment, the metabolite is MTXPG₃.

Where a level of MTXPGs is determined in a sample such as red blood cells (RBCs) or a cellular extract, the term “level” refers to the amount or concentration of at least one, two, three, four, five, six, seven or all of the MTXPG species in the sample. It is understood that a level can be an absolute level such as a molar concentration or weight or a relative level such as a percent or fraction compared to one or more other molecules in the sample. As a non-limiting example, a level of MTXPGs in RBCs can be expressed in terms of any unit known to one skilled in the art, including nmol/L RBCs, pmol/10⁹ RBCs, pmol/8×10⁸ RBCs, nmol/nmol hemoglobin, nmol/mg hemoglobin, pmol/25 mg hemoglobin, pmol/100 erythrocytes, and pmol/100 μl RBCs.

In one embodiment, a threshold MTXPG level (i.e.: the MTXPG level below which the BRM leads to unacceptable immunogenicity) is about 10-50 nmol/L red blood cells (RBC). In a further embodiment, the threshold MTXPG level is about 20-40 nmol/L red blood cells (RBC), or about 25 nmol/L red blood cells (RBC). Thus, in various exemplary embodiments:

(a) if the MTXPG level is at or below 25 nmol/L RBC, the methods may comprise:
(i) increasing a subsequent dose of MTX, or switching to an immunosuppressant other than MTX, to reduce immunogenicity caused by the BRM therapy; and/or
(ii) decreasing a subsequent dose of the BRM, or switching to a different BRM, to reduce immunogenicity caused by the BRM therapy; or

(b) if the MTXPG level is above 25 nmol/L RBC, the methods may comprise:
(i) maintaining or decreasing a subsequent dose of MTX; and/or
(ii) maintaining or increasing a subsequent dose of the BRM.

In embodiments wherein the immunosuppressant is leflunomide, the metabolite may be A 77 1726. In one embodiment, the threshold A77 1726 level is about 10-50 mg/L in plasma; in a further embodiment, the threshold A77 1726 level is about 20-40 mg/L plasma, or about 30 mg/L plasma. Thus, in various exemplary embodiments:

(a) if the A 77 1726 level is at or below 30 mg/L plasma, the methods may comprise:
(i) increasing a subsequent dose of leflunomide, or switching to an immunosuppressant other than leflunomide, to reduce immunogenicity caused by the BRM therapy; and/or
(ii) decreasing a subsequent dose of the BRM, or switching to a different BRM to reduce immunogenicity caused by the BRM therapy; or

(b) if the A 77 1726 level is above 30 mg/L plasma, the methods may comprise:
(i) maintaining or decreasing a subsequent dose of leflunomide; and/or
(ii) maintaining or increasing a subsequent dose of the BRM.

In embodiments wherein the immunosuppressant is azathiopurine/6-mercaptopurine, the metabolite may be 6 thioguanine or 6 methylmercaptopurine nucleotides. In one embodiment, the metabolite is 6 thioguanine and the threshold metabolite level is about 0.5 to about 5 μmol/L red blood cells (RBC); or about 1-3 μmol/L red blood cells (RBC); or about 1 μmol/L red blood cells (RBC). Thus, in various exemplary embodiments:

(a) If the 6 thioguanine level is at or below 1 μmol/L RBC, the methods may comprise:
(i) increasing a subsequent dose of azathiopurine, or switching to an immunosuppressant other than azathiopurine, to reduce immunogenicity caused by the BRM therapy; and/or
(ii) decreasing a subsequent dose of the BRM, or switching to a different BRM, to reduce immunogenicity caused by the BRM therapy; or

(b) If the 6 thioguanine level is above 1 μmol/L RBC, the methods may comprise:
(i) maintaining or decreasing a subsequent dose of azathiopurine; and/or
(ii) maintaining or increasing a subsequent dose of the BRM.

In another embodiment, the metabolite is 6 methylmercaptopurine and the threshold metabolite level is about 2 to 60 μmol/L red blood cells (RBC); or about 5-30 μmol/L red blood cells (RBC); or about 15 μmol/L red blood cells (RBC). Thus, in various exemplary embodiments:

(a) If the 6 methylmercaptopurine level is below 15 μmol/L RBC, the methods may comprise:
(i) increasing a subsequent dose of azathiopurine, or switching to an immunosuppressant other than azathiopurine, to reduce immunogenicity caused by the BRM therapy; and/or
(ii) decreasing a subsequent dose of the BRM, or switching to a different BRM, to reduce immunogenicity caused by the BRM therapy; or

(b) If the 6 methylmercaptopurine level is above 15 μmol/L RBC, the methods may comprise:
(i) maintaining or decreasing a subsequent dose of azathiopurine; and/or
(ii) maintaining or increasing a subsequent dose of the BRM.

The methods comprise administering to the subject a subsequent dose of immunosuppressant and/or a dose of the BRM in an amount effective to treat the subject based upon comparing the metabolite level in the sample to the threshold metabolite level. In one embodiment of all of the embodiments herein, the metabolite level is determined before the subject receives the BRM therapy. In this embodiment the dose of the immunosuppressant the subject received may be increased to achieve the threshold metabolite level before BRM therapy is initiated. Alternatively (or in addition), the dose of BRM to be administered may be decreased. In another embodiment of all of the embodiments herein, the metabolite level is determined after the subject receives BRM therapy, and either or both the immunosuppressant and the BRM therapy may be adjusted in response to the metabolite levels of the immunosuppressant. In various non-limiting embodiments, the methods are carried out on subjects that have been undergoing BRM therapy for at least 1 month, 2, months, 3 months, 4 months, 6 months, 12 months, 24 months, 29 months, or more.

In a further embodiment, the subject is being placed on BRM therapy or was placed on BRM therapy due to inadequate disease control when the subject was on immunosuppressant monotherapy. Similarly, the methods of the invention can be carried out on patients undergoing any type of BRM dosing regimen. In various non-limiting embodiments, the methods are carried out on subjects that are undergoing BRM therapy on a weekly basis, every other week, every 4 weeks, every 8 weeks, or any other dosing schedule at any suitable dose as determined by an attending physician.

In one embodiment, the BRM therapy comprises an anti-tumor necrosis factor (TNF) antibody, including but not limited to infliximab. In other embodiments, the BRM therapy comprises administering a therapeutic agent selected from the group consisting of infliximab, etanercept, adalimumab, golimumab, abatacept, rituximab, toclizumab, and ocrelizumab, natilizumab, epratuzumab, belimumab.

As will be apparent to those of skill in the art, the methods can be carried out repeatedly throughout a course of combined immunosuppressant/BRM co-therapy, to effect optimal treatment of the human subject while minimizing immunogenicity caused by the BRM therapy.

In one specific exemplary embodiment, the subject has rheumatoid arthritis, the immunosuppressant is MTX, and the BRM therapy is infliximab therapy. In this embodiment, the subject may be an adult subject, and may have been on MTX therapy for at least 5 years. In another embodiment, the subject may have been on infliximab therapy for between at least about 29 months. In a further embodiment, the MTX therapy may comprise a dosage of between about 10 mg to about 20 mg MTX on any suitable dosing schedule, such as once per week. In another embodiment, the infliximab therapy may comprise a dosage of between about 4.4 and about 8.4 mg/kg on any suitable dosing schedule, such as every 8 weeks.

In a second aspect, the present invention provides methods for optimizing dosage of an immunosuppressant comprising
(a) determining a metabolite level of an immunosuppressant in a human subject receiving or to receive a biological response modifier (BRM) therapy in combination with the immunosuppressant;
(b) comparing the metabolite level in the sample to a threshold metabolite level below which biological response modifier therapy leads to unacceptable immunogenicity; and
(c) recommending adjustment or adjusting a subsequent dose of immunosuppressant and/or biological response modifier based upon comparing the metabolite level in the sample to the threshold metabolite level.

The methods of the invention can be used, for example, in the clinical laboratory as a method to improve BRM therapy, by adjusting immunosuppressant and/or BRM doses to achieve a level of exposure commensurate with a low formation of autoantibodies to BRMs

The methods of this aspect of the invention involve recommending adjustment or adjusting a subsequent dose of immunosuppressant and/or biological response modifier based upon comparing the metabolite level in the sample to the threshold metabolite level. Thus, the method may result in a recommendation of a dose of the immunosuppressant and/or the BRM. Alternatively, the method may result in adjusting a dose (or adjusting dosing instructions) of the immunosuppressant and/or the BRM. Dosing instructions include, without limitation, lab results with preferred drug doses, data sheets, look-up tables setting forth preferred drug doses, instructions or guidelines for using the drug, package inserts to accompany the drug, professional medical advice and the like.

All embodiments and combinations of embodiments of the first aspect of the invention are equally applicable in this second aspect. In various further embodiments of this second aspect:
(a) If the metabolite level in the sample is below the threshold metabolite level, recommending comprises recommending an increase or increasing a subsequent dose of immunosuppressant to reduce immunogenicity caused by the BRM therapy.
(b) If the metabolite level in the sample is below the threshold metabolite level, recommending comprises recommending an increase or increasing a subsequent dose of the BRM to reduce immunogenicity caused by the BRM therapy.
(c) If the metabolite level in the sample is below the threshold metabolite level, recommending comprises recommending a change or changing the BRM therapeutic agent administered to the subject.

In various exemplary embodiments where the immunosuppressant is MTX and the metabolite is MTXPG, the methods of this second aspect may comprise:

(a) If the MTXPG level is at or below 25 nmol/L RBC, recommending comprises recommending an increase or increasing a subsequent dose of MTX to reduce immunogenicity caused by the BRM therapy;

(b) If the MTXPG level is at or below 25 nmol/L RBC, recommending comprises recommending a decrease or decreasing a subsequent dose of the BRM to reduce immunogenicity caused by the BRM therapy; or

(c) If the MTXPG level is at or below 25 nmol/L RBC, recommending comprises recommending a change or changing the BRM therapeutic agent administered to the subject.

In embodiments wherein the immunosuppressant is leflunomide and the metabolite is A 77 1726, the methods may comprise the following:

(a) If the A77 1726 level is at or below 30 mg/L plasma, recommending comprises recommending an increase or increasing a subsequent dose of leflunomide to reduce immunogenicity caused by the BRM therapy;

(b) If the A77 1726 level is at or below 30 mg/L plasma, recommending comprises recommending a decrease or decreasing a subsequent dose of the BRM to reduce immunogenicity caused by the BRM therapy; or

(c) If the A77 1726 level is at or below 30 mg/L RBC, recommending comprises recommending a change or changing the BRM therapeutic agent administered to the subject.

In embodiments wherein the immunosuppressant is azathiopurine/6-mercaptopurine and the metabolite is 6 thioguanine or 6 methylmercaptopurine nucleotides, the methods may comprise:

(a) If the 6 thioguanine level is at or below 1 μmol/L RBC, recommending comprises recommending an increase or increasing a subsequent dose of azathiopurine to reduce immunogenicity caused by the BRM therapy;

(b) If the 6 thioguanine level is at or below 1 μmol/L RBC, recommending comprises recommending a decrease or decreasing a subsequent dose of the BRM to reduce immunogenicity caused by the BRM therapy;

(c) If the 6 thioguanine level is at or below 1 μmol/L RBC, recommending comprises recommending a change or changing the BRM therapeutic agent administered to the subject.

(d) If the 6 methylmercaptopurine level is at or below 15 μmol/L RBC, recommending comprises recommending an increase or increasing a subsequent dose of azathiopurine to reduce immunogenicity caused by the BRM therapy;

(e) If the 6 methylmercaptopurine level is at or below 15 μmol/L RBC, recommending comprises recommending a decrease or decreasing a subsequent dose of the BRM to reduce immunogenicity caused by the BRM therapy; or

(f) If the 6 methylmercaptopurine level is at or below 15 μmol/L RBC, recommending comprises recommending a change or changing the BRM therapeutic agent administered to the subject.

### EXAMPLES

### Abstract

Methods:

Adult RA patients receiving weekly MTX with infliximab for more than three months were enrolled in a cross sectional study. Blood was collected at trough before the infusion of infliximab. Red blood cell (RBC) MTXPGs were measured using liquid chromatography while circulating levels of infliximab were measured using a cell based assay. ATIs were measured using enzyme immunoassays. Statistical analysis consisted of regression analyses and Wilcoxon tests.

Results:

In 61 patients enrolled ATIs were detected in 11 patients (18%). Regression analyses revealed that lower infliximab levels (median 3.3 μg/ml) were associated with the presence of ATI and lower RBC MTXPG levels (median 28 nmol/L) (p<0.05). Logistic regression revealed that RBC MTXPG₃ levels above 25 nmol/L were associated with a 4.7-fold lower likelihood of having ATI (OR=4.7; CI95%: 1.1-20.8; p=0.02). None of the 12 patients with RBC MTXPG above 50 nmol/L tested positive for ATI.

Conclusion:

These hypothesis generating data indicate that MTXPG impact infliximab exposure and ATI formation.

### Introduction

The introduction of anti-tumor necrosis factor-α (TNF-α) therapies including the chimeric monoclonal antibody infliximab has been a major advance in the treatment of rheumatoid arthritis (RA)¹. Yet, a large proportion of patients with RA do not respond adequately to this chimeric antibody. One of the most established mechanisms impairing the pharmacokinetic and clinical efficacy of infliximab is the emergence of immunogenicity and formation of neutralizing anti-drug antibodies or antibodies to Infliximab (ATIs)²⁻⁴. The consequences of this antidrug antibody mediated neutralization of infliximab is detrimental to the achievement of optimal serum levels producing adequate disease control, and shorter duration of response, secondary failure to treatment (i.e. loss result of efficacy) and shorter drug survival are observed. Furthermore, the formation of immune complexes between infliximab and ATIs increases the risk idiosyncratic reactions and appearance of infusion reactions^{2;5}. Currently, there is no possibility to predict which patient will mount an immune response against infliximab, although the schedule of administration⁶, dose intensity⁷, together with concomitant background MTX treatment⁷ can alter the propensity of infliximab to become immunogenic. However, the precise mechanism by which the combination of MTX with infliximab produce lower frequency of ATI formation compared to infliximab monotherapy is not established. In the present study we hypothesized that the activation of MTX to MTX polyglutamates (MTXPGs) may be in part responsible for the lower immunogenicity of infliximab in combination with MTX.

### Methods

The study was cross sectional study in adult RA patients at three study sites (Brigham and Women's, Boston, Mass.; The Center for Rheumatology, Albany, N.Y.; and Altoona Center for Clinical Research, Altoona Pa.). All RA patients (according to the 2010 ACR criteria for RA)⁸ enrolled had received a stable dose of MTX (for at least 4 months) in combination with infliximab for at least 3 months. Clinical parameters and demographic data were collected at the time of a single study visit. The study was approved by an independent ethic committee and informed consent was collected from all patients. Blood was drawn in EDTA containing tube and one SST tube at the time of the study visit. The blood was drawn just prior to the infliximab infusion (trough) and shipped overnight using refrigerated transportation kits. Disease activity was estimated using the clinical disease activity index (CDAI). Upon receipt of blood specimen, erythrocytes and serum were isolated and stored frozen at −80° C. RBC MTXPG₃ were measured using a liquid chromatography developed and validated in our laboratory⁹. Quantification limit was 5 nmol/L RBCs. Circulating levels of infliximab in serum were measured by Biomonitor (Copenhagen, DK) using a cell based assay consisting of erythroleukemic K562 cells transfected with a NFKB regulated firefly luciferase reporter-gene construct¹⁰. Antibodies to infliximab (ATIs) were measured using a proprietary enzyme immunoassay technique (Biomonitor, Copenhagen, Denmark). The cutoff for positive ATI was 0.9 U/L. Anti-citrullinated peptide auto-antibodies levels (Anti-MCV) were measured using ELISA (Orgentec Diagnostika, Mainz, Germany). Statistical analysis consists of multivariate logistic/linear regression and Wilcoxon tests as appropriate.

### Results

A total of 61 RA patients who received MTX with infliximab infusion were enrolled from June to December 2011. Patients characteristics are presented in Table I. In all patients the primary indication for infliximab infusion was high disease activity despite MTX treatment. At the time of the clinical assessment median RBC MTXPG₃ levels were 28 nmol/L (interquartile range 16-40 nmol/L) while median trough infliximab levels were 3.3 μg/ml (interquartile range 1.4-7.7 μg/ml). ATIs were detected in 11 patients (18%, ATI>0.9 U/L). As expected higher MTX dosages produced higher RBC MTXPG₃ levels (R²=0.19; p<0.01).

TABLE 1
Patients Characteristics.
Results are expressed as median (interquartile range) or
percentage as appropriate.

| | |
|---|---|
| Age (years) | 64 (54-71) |
| Gender (female, %) | 77% |
| Duration of disease (years) | 11 (6-20) |
| BMI | 27.4 (22.3-32.6) |
| Anti-MCV positivity (>20 Units) | 83.6% |
| Smokers (%) | 7 (12%) |
| Months of MTX therapy | 112 (65-150) |
| Methotrexate dose (mg/week) | 15 (10-20) |
| Infliximab dose (mg/Kg every 8 weeks) | 6.0 (4.4-8.4) |
| Duration of Infliximab Therapy (months) | 70 (29-107) |
| Previous anti-TNFs (%) | 36% |
| CDAI | 8 (4-13) |

BMI: Body mass index

Multivariate linear regression analyses revealed that lower infliximab trough levels were associated with lower infliximab doses, lower RBC MTXPG₃ levels, presence of ATI and shorter duration of MTX therapy (Table II).

TABLE II
Stepwise multivariate linear regression analysis for
infliximab trough levels.
Dependant variable was infliximab trough levels
expressed in μg/ml.

| | step | Global R² | Partial R² | Estimate | P value |
|---|---|---|---|---|---|
| Intercept | | | | 0.189 ± 2.1 | |
| infliximab | 1 | 0.364 | 0.364 | 3.48 ± 0.62 | <0.001 |
| (mg/Kg/8 weeks) | | | | | |
| ATI Positivity | 2 | 0.436 | 0.072 | −4.14 ± 1.85 | 0.029 |
| RBC MTXPG₃ | 3 | 0.469 | 0.033 | 0.08 ± 0.04 | 0.032 |
| (nmol/L) | | | | | |
| Duration MTX | 4 | 0.494 | 0.025 | −0.02 ± 0.01 | 0.100 |
| Therapy (Months) | | | | | |

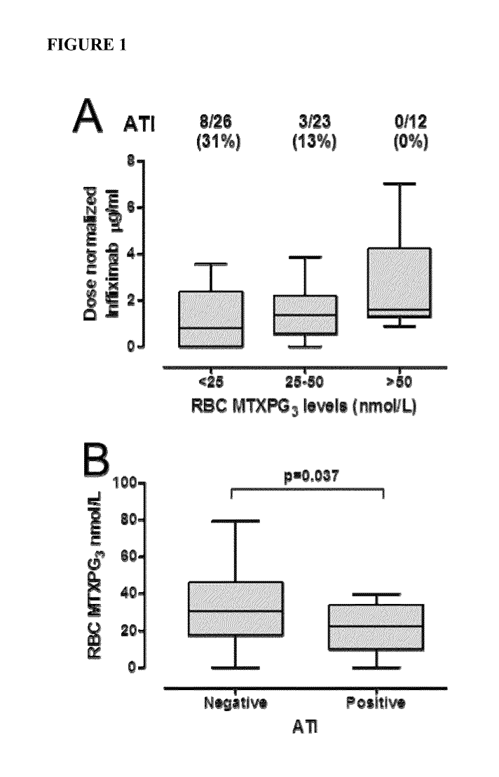

Nearly 50% of the variance in infliximab trough levels could be explained (Global R²=0.49). Patients with ATIs presented with several fold lower infliximab levels than those without antibodies (median 5.0 μg/mL [interquartile range: 2.0-8.4 μg/mL] vs. <0.5 μg/mL [interquartile range: <0.5-1.4 μg/mL]; p<0.001). Similar infliximab doses were administered in ATI negative (median 6.0 mg/Kg/8 weeks [interquartile range: 4.2-8.4 mg/Kg/8 weeks]) and positive patients (median 6.0 mg/Kg/8 weeks [interquartile range: 4.8-9.0 mg/Kg/8 weeks]) (p>0.9). There was no difference in MTX doses between ATI positive (median 17.5 mg/week [interquartile range: 10-20 mg/week]) and ATI negative patients (median 13.5 mg/week [interquartile range: 7.5-17.5 mg/week])(p=0.22). However, patients having ATIs presented lower RBC MTXPG₃ (median 22 nmol/L) than patients without ATI (median 30 nmol/L) (p=0.037) (FIG. 1B). Logistic regression revealed that RBC MTXPG₃ levels above 25 nmol/L were associated with a 4.7-fold lower likelihood of having ATI (OR=4.7; CI95%: 1.1-20.8; p=0.02) and none of the 12 patients with RBC MTXPG₃ above 50 nmol/L tested positive for ATI (FIG. 1A). Alternatively, 3 out of 4 patients (75%) with RBC MTXPG₃ levels below 5 nmol/L presented with ATI. Circulating levels of infliximab, presence of ATI, BMI, Anti-MCV, RBC MTXPGs levels and smoking status were not associated with the disease activity as assessed using the CDAI (p>0.11; data not shown).

### Discussion

In the past few years a wealth of data have established that inter-individual variations in serum levels of infliximab and ATI positivity are associated with variable therapeutic response to infliximab in RA^{3;11;12}. While the determination of these pharmacokinetic and exposure markers are not currently standard of care, their potential for therapeutic decision making and individualization of infliximab dosing is under intense debate. This interest is further justified by the necessity to control costs and improve long term disease control in RA.

The MTX prodrug remains the cornerstone front line treatment for RA and the most commonly small molecule disease modifiers prescribed with anti-TNFs such as infliximab. Our report indicates that concomitant MTX is beneficial to infliximab pharmacokinetics and is the first to suggest that elevated RBC MTXPG levels maximize infliximab systemic exposure and lower the incidence of ATI formation. While not being bound by any specific mechanism of action, it is hypothesized that the potent effect of MTXPGs on AICAR transformylase followed by de novo purine biosynthesis inhibition and suppression of T cell clonality or delayed T cell repopulation may explain the propensity of MTXPG to suppress infliximab immunogenicity.

It should also be recognized that all patients enrolled in the study were receiving infliximab because of inadequate disease control despite MTX monotherapy, and that the primary cause for the lack of MTX efficacy may have resulted from poor MTXPG exposure (e.g. <25 nmol/L) or alternatively because of adequate MTXPG accumulation in the context of disease refractoriness to the antifolate effect (e.g. <50 nmol/L). As such, the observation that none of the patients having MTXPG above 50 nmol/L were positive for ATI suggests that the lack of anti-inflammatory effects does not preclude the achievement of adequate immunosuppression to promote tolerance to infliximab. Alternatively, the indication that 75% of patients with MTXPG below 5 nmol/L had ATI suggests that poor absorption of MTX or non-compliance to background MTX therapy may be important to maximizing infliximab exposure.

The present studies suggest that controlling background MTX therapy and achieving adequate MTX exposure can potentiate efficacy of infliximab in RA in addition to perhaps facilitating infliximab dosage reduction strategies.

In conclusion, these hypothesis generating data indicate that MTX exposure impact infliximab pharmacokinetics and formation of ATI in RA.

### REFERENCES

(1) Maini R, St Clair E W, Breedveld F, Furst D, Kalden J, Weisman M et al. Infliximab (chimeric anti-tumour necrosis factor alpha monoclonal antibody) versus placebo in rheumatoid arthritis patients receiving concomitant methotrexate: a randomised phase III trial. ATTRACT Study Group. Lancet 1999; 354(9194):1932-1939.
(2) Baert F, Noman M, Vermeire S, Van Assche G, D' Haens G, Carbonez A et al. Influence of Immunogenicity on the Long-Term Efficacy of Infliximab in Crohn's Disease. The New England Journal of Medicine 2003; 348(7):601-608.
(3) Wolbink G J, Vis M, Lems W, Voskuyl A E, de G E, Nurmohamed M T et al. Development of antiinfliximab antibodies and relationship to clinical response in patients with rheumatoid arthritis. Arthritis Rheum 2006; 54(3):711-715.
(4) Bendtzen K, Geborek P, Svenson M, Larsson L, Kapetanovic M C, Saxne T. Individualized monitoring of drug bioavailability and immunogenicity in rheumatoid arthritis patients treated with the tumor necrosis factor alpha inhibitor infliximab. Arthritis Rheum 2006; 54(12):3782-3789.
(5) Pascual-Salcedo D, Plasencia C, Ramiro S, Nu+¦o L, Bonilla G, Nagore D et al. Influence of immunogenicity on the efficacy of long-term treatment with infliximab in rheumatoid arthritis. Rheumatology 2011.
(6) Hanauer S B, Wagner C L, Bala M, Mayer L, Travers S, Diamond R H et al. Incidence and importance of antibody responses to infliximab after maintenance or episodic treatment in Crohn's disease. Clin Gastroenterol Hepatol 2004; 2(7):542-553.
(7) Maini R N, Breedveld F C, Kalden J R, Smolen J S, Davis D, Macfarlane J D et al. Therapeutic efficacy of multiple intravenous infusions of anti-tumor necrosis factor alpha monoclonal antibody combined with low-dose weekly methotrexate in rheumatoid arthritis. Arthritis Rheum 1998; 41(9):1552-1563.
(8) Neogi T, Aletaha D, Silman A J, Naden R L, Felson D T, Aggarwal R et al. The 2010 American College of Rheumatology/European League Against Rheumatism classification criteria for rheumatoid arthritis: Phase 2 methodological report. Arthritis Rheum 2010; 62(9):2582-2591.
(9) Dervieux T, Orentas L D, Marcelletti J, Pischel K, Smith K, Walsh M et al. HPLC determination of erythrocyte methotrexate polyglutamates after low-dose methotrexate therapy in patients with rheumatoid arthritis. Clin Chem 2003; 49(10):1632-1641.
(10) Lallemand C, Kavrochorianou N, Steenholdt C, Bendtzen K, Ainsworth M A, Meritet J F et al. Reporter gene assay for the quantification of the activity and neutralizing antibody response to TNFalpha antagonists. J Immunol Methods 2011; 373(1-2):229-239.
(11) St Clair E W, Wagner C L, Fasanmade A A, Wang B, Schaible T, Kavanaugh A et al. The relationship of serum infliximab concentrations to clinical improvement in rheumatoid arthritis: results from ATTRACT, a multicenter, randomized, double-blind, placebo-controlled trial. Arthritis Rheum 2002; 46(6):1451-1459.
(12) Mulleman D, Meric J C, Paintaud G, Ducourau E, Magdelaine-Beuzelin C, Valat J P et al. Infliximab concentration monitoring improves the control of disease activity in rheumatoid arthritis. Arthritis Res Ther 2009; 11(6):R178.
(13) Morgan S L, Oster R A, Lee J Y, Alarcon G S, Baggott J E. The effect of folic acid and folinic acid supplements on purine metabolism in methotrexate-treated rheumatoid arthritis. Arthritis Rheum 2004; 50(10):3104-3111.
(14) Genestier L, Paillot R, Fournel S, Ferraro C, Miossec P, Revillard J P. Immunosuppressive properties of methotrexate: apoptosis and clonal deletion of activated peripheral T cells. J Clin Invest 1998; 102(2):322-328.
(15) Joseph A, Neff K, Richard J, Gao L, Bangari D, Joly M et al. Transient low-dose methotrexate induces tolerance to murine anti-thymocyte globulin and together they promote long-term allograft survival. J Immunol 2012; 189(2):732-743.
(16) Krieckaert C L, Nurmohamed M T, Wolbink G J. Methotrexate reduces immunogenicity in adalimumab treated rheumatoid arthritis patients in a dose dependent manner Ann Rheum Dis 2012.

I claim:

1. A method for treating a human subject in need of combined immunosuppressant and biological response modifier (BRM) therapy, comprising
(a) determining a metabolite level of an immunosuppressant in a sample from a human subject receiving or to receive a BRM therapy in combination with the immunosuppressant;
(b) comparing the metabolite level in the sample to a threshold metabolite level below which the BRM leads to unacceptable immunogenicity; and
(c) administering to the human subject a subsequent dose of immunosuppressant and/or a dose of the BRM in an amount effective to treat the subject based upon comparing the metabolite level in the sample to the threshold metabolite level.

2. The method of claim 1, wherein the BRM therapy comprises administering an anti-tumor necrosis factor (TNF) antibody.

3. The method of claim 1 wherein the BRM therapy comprises administering infliximab.

4. The method of claim 3, wherein the immunosuppressant is methotrexate (MTX).

5. The method of claim 4, wherein the metabolite is methotrexate polyglutamate MTXPG₃.

6. The method of claim 5, wherein the sample is a red blood cell (RBC) sample.

7. The method of claim 6, wherein if the MTXPG₃ level is at or below 25 nmol/L RBC,
(i) increasing a subsequent dose of MTX or switching to an immunosuppressant other than MTX; and/or
(ii) decreasing a subsequent dose of infliximab.

8. The method of claim 6, wherein if the MTXPG₃ level is above 25 nmol/L RBC,
(i) maintaining or decreasing a subsequent dose of MTX; and/or
(ii) maintaining or increasing a subsequent dose of infliximab.

9. The method of claim 7 wherein the subject has rheumatoid arthritis.

10. The method of claim 8 wherein the subject has rheumatoid arthritis.

11. A method for optimizing dosage of an immunosuppressant comprising
(a) determining a metabolite level of an immunosuppressant in a sample from a human subject receiving or to receive a BRM therapy in combination with the immunosuppressant;
(b) comparing the metabolite level in the sample to a threshold metabolite level below which the BRM therapy leads to unacceptable immunogenicity; and
(c) recommending adjustment or adjusting a subsequent dose of immunosuppressant and/or biological response modifier to be administered to the human subject based upon comparing the metabolite level in the sample to the threshold metabolite level.

12. The method of claim 11, wherein the immunosuppressant is MTX.

13. The method of claim 12, wherein the metabolite is MTXPG.

14. The method of claim 11, wherein the immunosuppressant is leflunomide.

15. The method of claim 14, wherein the metabolite is A77 1726.

16. The method of claim 11, wherein the immunosuppressant is azathiopurine.

17. The method of claim 16, wherein the metabolite is 6-thioguanine nucleotide or 6-methylmercaptopurine.

18. The method of claim 12, wherein the BRM therapy comprises administration of a therapeutic agent selected from the group consisting of infliximab, etanercept, adalimumab, golimumab, abatacept, rituximab, toclizumab, and ocrelizumab, natalizimab, belimumab.

19. The method of claim 14, wherein the BRM therapy comprises administration of a therapeutic agent selected from the group consisting of infliximab, etanercept, adalimumab, golimumab, abatacept, rituximab, toclizumab, and ocrelizumab, natalizimab, belimumab.

20. The method of claim 16, wherein the BRM therapy comprises administration of a therapeutic agent selected from the group consisting of infliximab, etanercept, adalimumab, golimumab, abatacept, rituximab, toclizumab, and ocrelizumab, natalizimab, belimumab.
